(19) United States
(12) Patent Application Publication
(10) Pub. No.: US 2005/0147584 A1
(43) Pub. Date: Jul. 7, 2005

(54) Compositions and methods comprising memantine and polyanionic polymers

(75) Inventors: Patrick M. Hughes, Aliso Viejo, CA (US); Orest Olejnik, Coto De Caza, CA (US)

Correspondence Address: BRENT A. JOHNSON;ALLERGAN, INC., T2-7H, 2525 Dupont Drive, Irvine, CA, 92612, US

(73) Assignee: Allergan, Inc.

(21) Appl. No.: 10/752,125

(22) Filed: Jan. 5, 2004

(51) Int. Cl. A61K 38/17; A61K 31/728; A61K 31/717; A61K 31/716; A61K 31/785

(52) U.S. Cl. 424/78.31; 514/12; 514/54; 514/57; 514/662; 514/60

(57) ABSTRACT

Disclosed herein are aqueous solutions comprising a neuroprotective amine related to adamantane and a polyanionic polymer. Also disclosed herein are methods of treating glaucoma and methods of treating a disease or a condition wherein migration or proliferation of retinal pigment epithelium or glial cells causes or contributes to the cause of said disease or condition.

### FIELD OF THE INVENTION

This invention relates to pharmaceutical compositions. More particularly, this invention relates to compositions comprising adamantane-based neuroprotective amines.

### BACKGROUND OF THE INVENTION

1. Description of the Related Art

Glaucoma is a disease of the eye characterized by increased intraocular pressure. On the basis of its etiology, glaucoma has been classified as primary or secondary. For example, primary glaucoma in adults (congenital glaucoma) may be either open-angle or acute or chronic angle-closure. Secondary glaucoma results from pre-existing ocular diseases such as uveitis, intraocular tumor or an enlarged cataract.

The underlying causes of primary glaucoma are not yet known. The increased intraocular tension is due to the obstruction of aqueous humor outflow. In chronic open-angle glaucoma, the anterior chamber and its anatomic structures appear normal, but drainage of the aqueous humor is impeded. In acute or chronic angle-closure glaucoma, the anterior chamber is shallow, the filtration angle is narrowed, and the iris may obstruct the trabecular meshwork at the entrance of the canal of Schlemm. Dilation of the pupil may push the root of the iris forward against the angle, and may produce pupilary block and thus precipitate an acute attack. Eyes with narrow anterior chamber angles are predisposed to acute angle-closure glaucoma attacks of various degrees of severity.

Secondary glaucoma is caused by any interference with the flow of aqueous humor from the posterior chamber into the anterior chamber and subsequently, into the canal of Schlemm. Inflammatory disease of the anterior segment may prevent aqueous escape by causing complete posterior synechia in iris bombe, and may plug the drainage channel with exudates. Other common causes are intraocular tumors, enlarged cataracts, central retinal vein occlusion, trauma to the eye, operative procedures and intraocular hemorrhage.

Considering all types together, glaucoma occurs in about 2% of all persons over the age of 40 and may be asymptotic for years before progressing to rapid loss of vision. Several topical ophthalmic therapeutic agents are currently administered to patients in an effort to reduce intraocular pressure, including prostaglandins and prostamides, α₂-adrenergic agonists, β-adrenergic antagonists, and others.

In addition to intraocular pressure reduction, a complimentary approach to the treatment of the sequelae of glaucoma is the administration of neuroprotective agents. Glaucoma is associated with an increase in the rate of retinal ganglion cell loss, resulting in vision loss. U.S. Pat. No. 6,482,854 and Sugrue (Journal of Medicinal Chemistry, 1997, Vol. 40, No. 18, 2793-2809) teach the use of neuroprotective agents to treat glaucoma. While the exact mechanism of these neuroprotective agents may not be unambiguously established, it is believed that these compounds work as glutamate antagonists. Retinal ganglion cells, like other ganglion cells, have surface receptors for glutamate as well as other amino acids, which trigger neuronal excitation. However, excess amino acid associated neuroexcitation causes neuronal degeneration and cell death. In the case of glaucoma, vitreous concentrations of glutamate are double that of a healthy individual, and thus it is believed that the excess glutamate causes accelerated ganglion cell loss and accompanying loss of vision. There are several types of glutamate receptors which are classified based on their function and mechanism of action. One class of glutamate receptors, the ionotropic receptors, works through Ca²⁺-specific ion channels. This class can be divided into subclasses based upon their selective agonists. It is believed that memantine and other adamantane-based amines act as antagonists to one of these subclasses of receptors, referred to as the N-methyl-D-aspartate (NMDA) receptor according to the name of its selective agonist. Thus, memantine and other adamantane-based amines counteract glutamate neuroexcitotocity, and retard vision loss in glaucoma sufferers.

In addition to the treatment of glaucoma, it is believed that memantine and other adamantane-based glutamate antagonists are useful in the treatment of other diseases. U.S. Pat. No. 6,573,280 and U.S. Pat. No. 5,922,773 incorporated herein by reference, teach that glutamate causes migration and proliferation of retinal pigment epithelium and/or glial cells, and is thus useful in treating proliferative vitreoretinopathy.

### BRIEF DESCRIPTION OF THE INVENTION

Disclosed herein are compositions comprising memantine and a polyanionic polymer, as well as methods and products related thereto.

### BRIEF DESCRIPTION OF THE DRAWING FIGURES

FIG. 1 is a plot of the permeability of carboxymethylcellulose (CMC) and non-CMC formulations of memantine through dialysis membranes.

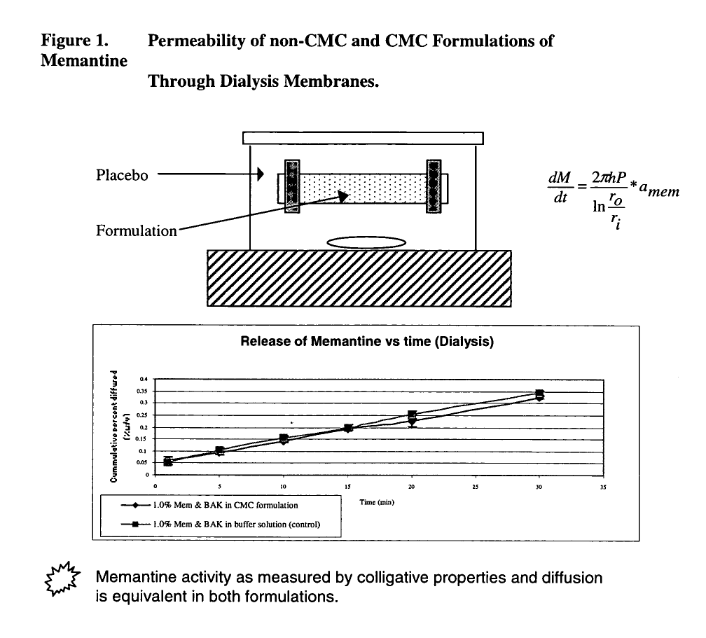

### DETAILED DESCRIPTION OF THE INVENTION

While not intending to limit the scope of the invention in any way, or to be bound or limited in any way by theory, we have surprisingly discovered that polyanionic polymers, when used in conjunction with an adamantane-based amine, can help to attenuate certain adverse effects associated with the topical ophthalmic use of said amine.

Disclosed herein are aqueous solutions comprising an adamantane-based neuroprotective amine and a polyanionic polymer.

Also disclosed herein is a method which comprises administering an effective amount of neuroprotective compound comprising an adamantyl moiety and an amine moiety in an aqueous composition comprising an effective amount of a soluble polyanionic polymer to the eye of a mammal suffering from glaucoma, ocular hypertension, nystagmus, proliferative vitreal retinopathy or ocular neurodegenerative diseases.

Also disclosed herein is an eye drop product comprising an aqueous solution comprising an effective amount of an adamantane-based neuroprotective amine and an effective amount of a polyanionic polymer, and a package for dispensing said solution in the form of drops suitable for administration to an eye of a mammal.

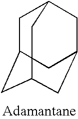

An adamantane-based amine is a compound having an amine which is directly or indirectly bonded to or coupled with an adamantane. In other words, the adamantane may be directly bonded to the nitrogen of the amine, or a linking group consisting of one or more atoms may connect the adamantane to the amine. Additionally, the adamantane may have additional substituents, such as a methyl group or a small alkyl group, attached. A group comprising the basic cage structure of adamantane and one or more substituents is referred to as an “adamantyl” moiety. The term “amine” should be understood as being broadly applied to both a molecule, or a moiety or functional group, as generally understood in the art, and may be primary, secondary, or tertiary. A neuroprotective compound is a compound which is generally understood in the art to reduce the rate of ganglion cell loss in a neurodegenerative disease or condition such as Alzheimer's disease or glaucoma. While not intending to limit the scope of the invention in any way, three compounds which are adamantane based neuroprotective-amines, and are also neuroprotective compounds comprising an adamantyl moiety and an amine moiety, are amantadine, rimantadine, and memantine.

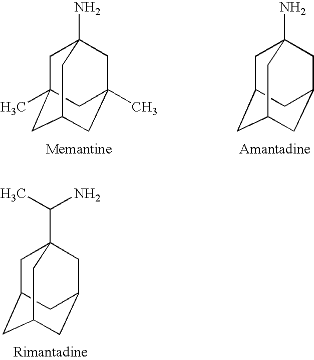

The terms “memantine”, “amantadine”, and “rimantadine” as used herein refer to the free base forms of the amine, or any of the various salts, such as memantine hydrochloride, which can be prepared by the addition of an acid to the free base. The determination of the amount of memantine used in the compositions disclosed herein is well within the ability of one having ordinary skill in the art. An “effective” amount of memantine is an amount which has a detectable effect over a similar composition or method which comprises no memantine or any other active ingredient which would be expected to have an effect similar to that of memantine.

In referring to concentrations of memantine herein, the numeric value for the concentration is understood to be the concentration of the free base, regardless of the form in which the memantine is used. Since there is a large range of concentrations or amounts at which memantine is effective, the concentration or amount of memantine as used herein may vary. One embodiment comprises from 0.05 to 5% memantine. Other embodiments comprise from 0.05% to 2% memantine. Some compositions comprise from 0.05% to 2.5% memantine. Another composition comprises from 0.2% to 3% memantine. Some compositions comprise from 0.1 to 2% memantine. Other compositions comprise from 0.5% to 2% memantine. Another embodiment comprises from 0.5% to 3.5% memantine. Other embodiments comprise from 0.3% to 1.5%. Another composition comprises from 0.5% to 1.3% memantine. Other embodiments comprise from 0.1% to 1% memantine. Another embodiment comprises from about 0.5% to about 1% memantine. Other composition comprise about 0.5% memantine. Other compositions comprise about 1% memantine.

The term “polyanionic polymer” refers, in the broadest sense understood in the art, to a polymer comprising several anionic moieties. While not intending to limit the scope of the invention in any way, typical examples of polyanionic polymers are carboxymethylcellulose, hyaluronic acid, carboxymethylamylose, anionic polymers derived from acrylic acid (meaning to include polymers from acrylic acid, acrylates and the like and mixtures thereof), anionic polymers derived from methacrylic acid (meaning to include polymers from methacrylic acid, methacrylates, and the like and mixtures thereof), poly(methacrylic acid) derivatives, polyphospazene derivatives, poly(aspartic acid), anionic polymers of amino acids (meaning to include polymers of amino acids, amino acid salts, and the like and mixtures thereof), acidic gelatin, and anionic polymers derived from alginic acid (meaning to include alginic acid, alginates, and the like and mixtures thereof). In one embodiment, the polyanionic polymer comprises carboxymethylcellulose. Carboxymethylcellulose is a polyanionic species, and thus may have one or more countering cations, by which it may be referred. For example, sodium carboxymethylcellulose refers to a carboxymethylcellulose having sodium as the counterion.

The term “soluble”, in reference to a polyanionic polymer, means that said polymer dissolves in an aqueous solution at an effective concentration.

An “effective” amount or concentration of a polyanionic polymer is an amount which has a detectable effect over a similar composition or method which comprises no polyanionic polymer or any other component which would be expected to have an effect similar to that of polyanionic polymer. Since there is a large range of concentrations of the polyanionic polymers that are effective, the concentration of the polyanionic polymer may vary significantly in the compositions and methods disclosed herein. One composition comprises from 0.1 to 5% carboxymethylcellulose. Another composition comprises 0.1% to 5% sodium carboxymethylcellulose. Other embodiments comprise 0.4% to 4.5% sodium carboxymethylcellulose. Another composition comprises from 0.5% to 4% sodium carboxymethylcellulose. Another composition comprises from 0.1% to 1% sodium carboxymethylcellulose. Other compositions comprise from 0.1% to 1.5% carboxymethylcellulose. Another embodiment comprises from 0.3% to 0.8% carboxymethylcellulose. Other embodiments comprise from 0.4% to 1.5% sodium carboxymethylcellulose. Still other compositions comprise from 0.5% to 2% sodium carboxymethylcellulose. Another embodiment comprises about 0.5% sodium carboxymethylcellulose.

Preservatives may also be used to prevent bacterial contamination in multiple-use ophthalmic preparations. Cationic, anionic, and nonionic preservatives may be used, and while not intending to be limiting, examples include benzalkonium chloride, stabilized oxychloro complexes (otherwise known as Purite®), phenylmercuric acetate, chlorobutanol, benzyl alcohol, parabens, and thimerosal.

Unexpectedly, certain compositions disclosed herein comprise a cationic preservative. While not intending to limit the scope of the invention, or be bound in any way by theory, it is generally expected that cationic preservatives will form an insoluble complex with the polyanionic polymer, which precipitates from solution. However, the compositions prepared in Example 1 contain a cationic preservative, and no insoluble material formed during or subsequent to the preparations. Thus, while not intending to limit the scope of the invention, the drug-polyanionic polymer combinations disclosed herein have an added advantage of flexibility in terms of the use of preservatives. Quaternary ammonium salts, such as benzalkonium chloride, are common cationic preservatives.

An “effective” amount or concentration of a preservative is the concentration required to significantly reduce the microbial contamination of a composition, relative to a similar composition that does not contain a component which can inhibit the growth of or kill microbes. Since an effective amount or concentration encompasses a large range of values, the concentration or amount of the cationic preservative used herein may vary significantly. In one embodiment, from 10 ppm to 200 ppm benzalkonium chloride is used. Another composition comprises, about 20 ppm benzalkonium chloride.

In addition to being useful in the treatment of glaucoma, memantine and other adamantane-based compounds glutamate antagonists can be used to reduce or control retinal pigment epithelium and/or glial migration and the diseases or conditions related thereto. Thus, the compositions disclosed herein can be used to treat a disease or condition wherein migration or proliferation of retinal pigment epithelium or glial cells causes or contributes to the cause of said disease or condition. The relationship may be direct or indirect, and the migration or proliferation retinal pigment epithelium or glial cells may be a root cause of said disease or condition, or may be a symptom of another underlying disease or condition. While not intending to limit the scope of the invention in any way, the following are examples of the types of diseases or conditions treated by the disclosed method: non-exudative age related macular degeneration, exudative age related macular degeneration, choroidal neovascularization, acute macular neuroretinopathy, cystoid macular edema, diabetic macular edema, Behcet's disease, diabetic retinopathy, retinal arterial occlusive disease, central retinal vein occlusion, uveitic retinal disease, retinal detachment, trauma, conditions caused by laser treatment, conditions caused by photodynamic therapy, photocoagulation, radiation retinopathy, epiretinal membranes, proliferative diabetic retinopathy, branch retinal vein occlusion, anterior ischemic optic neuropathy, non-retinopathy diabetic retinal dysfunction, and retinitis pigmentosa.

Other specific embodiments are contemplated herein, which are a combination of the aforementioned embodiments. Those skilled in the art will also recognize that additional embodiments may also be prepared by combining the aforementioned embodiments, which would also be considered to be within the scope and spirit of the present invention.

One composition comprises memantine and sodium carboxymethylcellulose.

Another embodiment comprises from 0.05% to 2% memantine and from 0.1% to 5% sodium carboxymethylcellulose.

Another embodiment comprises from 0.05% to 2.5% memantine, from 0.1% to 5% sodium carboxymethylcellulose, and from 10 ppm to 200 ppm benzalkonium chloride.

Another composition comprises from about 0.5% to about 2% memantine, about 0.5% sodium carboxymethylcellulose, and about 20 ppm benzalkonium chloride.

Another composition comprises about 1% memantine, from 0.1% to 1.5% carboxymethylcellulose, and said composition further comprises an effective amount of benzalkonium chloride.

Other compositions comprise from 0.2% to 3% memantine, from 0.5% to 4% sodium carboxymethylcellulose, and an effective amount of benzalkonium chloride.

Another composition comprises about 1% memantine, from 0.1% to 1.5% carboxymethylcellulose, and further comprises an effective amount of benzalkonium chloride.

Another composition comprises about 0.5% memantine, from 0.1% to 1.5% carboxymethylcellulose, and further comprises an effective amount of benzalkonium chloride.

One composition comprises from 0.1% to 1% memantine, from 0.1% to 1% sodium carboxymethylcellulose, and from 10 ppm to 200 ppm benzalkonium chloride.

Another embodiment comprises from about 0.5% to about 1% memantine, about 0.5% sodium carboxymethylcellulose, and about 20 ppm benzalkonium chloride.

Other compositions comprise about 0.5% memantine, from 0.3% to 0.8% carboxymethylcellulose, and further comprise an effective amount of benzalkonium chloride.

Other embodiments comprise from 0.5% to 2% memantine, an effective amount of carboxymethylcellulose, and further comprise an effective amount of benzalkonium chloride.

Other compositions comprise about 1% memantine, from 0.3% to 0.8% carboxymethylcellulose, and an effective amount of benzalkonium chloride.

Other aqueous solutions comprise from 0.3% to 1.5% memantine, from 0.5% to 2% sodium carboxymethylcellulose, and an effective amount of benzalkonium chloride.

Another aqueous solution comprises from 0.5% to 1.3% memantine, from 0.4% to 1.5% sodium carboxymethylcellulose, and 20 ppm benzalkonium chloride.

Another composition comprises from 0.5% to 3.5% memantine, from 0.4% to 4.5% sodium carboxymethylcellulose, and 20 ppm benzalkonium chloride.

While not intending to limit the scope of the invention in any way, it is often useful to include a buffer in ophthalmic compositions to maintain the pH from about 6 to about 8 for optimal comfort. Buffers used are those known to those skilled in the art, and, while not intending to be limiting, some examples are acetate, borate, carbonate, citrate, and phosphate buffers. Tonicity agents such as glycerin, mannitol, sorbitol, sodium chloride, and other electrolytes may also be used in ophthalmic compositions to adjust the concentration of dissolved material to the desired isotonic range. Surfactants such as polysorbates, poloxamers, alcohol ethoxylates, ethylene glycol-propylene glycol block copolymers, fatty acid amides, alkylphenol ethoxylates, or phospholipids may also be used in ophthalmic compositions. Chelating agents may also be used in ophthalmic compositions to enhance preservative effectiveness. While not intending to be limiting, some useful chelating agents are edetate salts, like edetate disodium, edetate calcium disodium, edetate sodium, edetate trisodium, and edetate dipotassium. The foregoing discussion of compounds typically used in ophthalmic compositions is given purely for purposes of example, to more readily enable a person of ordinary skill in the art to carry out the methods disclosed herein, and is not intended to limit the scope of the invention in any way.

### EXAMPLE 1

Memantine, 1 -amino-3,5-dimethyladamantane hydrochloride (memantine HCl), was formulated in a standard aqueous vehicle and a vehicle containing 0.5% sodium carboxymethylcellulose (CMC) (Aqualon Type 7LFH, MW 90 kDa) with 20 ppm benzalkonium chloride (BAK) (Tables 1 and 2). The compositions of Table 1 were prepared by methods commonly used in the art.

TABLE 1
Non-CMC Memantine HCl Formulations

| Components | Function | Percent w/v | | | | | |
|---|---|---|---|---|---|---|---|
| Memantine | Active | 0.05 | 0.1 | 0.2 | 0.5 | 0.75 | 1.0 |
| HCl | | | | | | | |
| Sodium | Tonicity | 0.46 | 0.46 | 0.46 | 0.36 | 0.30 | 0.23 |
| Chloride | adjuster | | | | | | |
| Boric Acid | Buffering | 0.64 | 0.64 | 0.64 | 0.64 | 0.64 | 0.64 |
| | agent | | | | | | |
| Sodium Borate, | Buffering | 0.16 | 0.16 | 0.16 | 0.16 | 0.16 | 0.16 |
| Decahydrate | agent | | | | | | |
| Benzalkonium | Preservative | 0.002 | 0.002 | 0.002 | 0.002 | 0.002 | 0.002 |
| Chloride | | | | | | | |
| Hydrochloric | pH | 7.4 | 7.4 | 7.4 | 7.4 | 7.4 | 7.4 |
| Acid | adjustment | | | | | | |
| Sodium | pH | 7.4 | 7.4 | 7.4 | 7.4 | 7.4 | 7.4 |
| Hydroxide | adjustment | | | | | | |
| Purified Water | Vehicle | Q.S. | Q.S. | Q.S. | Q.S. | Q.S. | Q.S. |

The compositions of Table 2 were prepared according to the following procedure. Two aqueous phases designated Part I and Part II respectively, were separately prepared.

Part I

Purified water (2000 mL) was charged to a vessel and mixing was initiated, and sodium carboxymethylcellulose (20 g) was then added and mixed until dispersed.

Part II

Purified water (1700 mL) was charged a vessel and mixing is initiated.

Sodium chloride (20.0 g), potassium chloride (5.6 g), sodium lactate (20 ml, 60% solution), calcium chloride (0.80 g), magnesium chloride (0.24 g), and benzalkonium chloride (20 mL) were sequentially added allowing each to dissolve before adding the next. Memantine HCl (4.0 g for the 0.1% w/v formulation) was then added with mixing until dissolved.

After the two aqueous phases were prepared, (Part II) was transferred into the bulk phase (Part I) in the main batch vessel while mixing, and the mixture was thoroughly mixed for 15 minutes. Sodium Hydroxide or Hydrochloric acid was used to adjust the pH to 6.4-6.6. Water was then added to bring the batch volume to 4000 ml and the pH was adjusted to 6.4-6.6 with 1 N NaOH or 1 N HCl if necessary. The solution was then mixed thoroughly for 20 to 30 minutes, and sterile filtered with a Suporlife DCF CHS92DSPPK 0.2 μm filter. A 500 ml filter flush of the Memantine HCl Topical Solution was required.

TABLE 2
CMC-Memantine HCl Formulations

| Components | Function | Percent w/v | | | | |
|---|---|---|---|---|---|---|
| Memantine HCl | Active | 0.1 | 0.2 | 0.5 | 0.75 | 1.0 |
| Sodium Chloride | Tonicity | 0.50 | 0.48 | 0.40 | 0.33 | 0.26 |
| | adjuster | | | | | |
| Potassium Chloride | Electrolyte | 0.14 | 0.14 | 0.14 | 0.14 | 0.14 |
| Sodium Lactate | Electrolyte | 0.3 | 0.3 | 0.3 | 0.3 | 0.3 |
| Calcium Chloride, | Electrolyte | 0.02 | 0.02 | 0.02 | 0.02 | 0.02 |
| dihydrate | | | | | | |
| Magnesium Chloride, | Electrolyte | 0.006 | 0.006 | 0.006 | 0.006 | 0.006 |
| hexahydrate | | | | | | |
| Benzalkonium | Preservative | 0.002 | 0.002 | 0.002 | 0.002 | 0.002 |
| Chloride | | | | | | |
| Sodium CMC | Vicosifier | 0.5 | 0.5 | 0.5 | 0.5 | 0.5 |
| (Type 7LFH) | | | | | | |
| Hydrochloric Acid | pH | 6.5 | 6.5 | 6.5 | 6.5 | 6.5 |
| | adjustment | | | | | |
| Sodium Hydroxide | pH | 6.5 | 6.5 | 6.5 | 6.5 | 6.5 |
| | adjustment | | | | | |
| Purified Water | Vehicle | Q.S. | Q.S. | Q.S. | Q.S. | Q.S. |

The effect of a polyanionic polymer on the tolerability of an adamantane-based neuroprotective amine was assessed using sodium carboxymethylcellulose (CMC) as the model polyanionic polymer, and memantine hydrochloride (pKa 10.27) as the model neuroprotective adamantane-based amine.

While not intending to limit the scope of the invention, or be bound in any way by theory, it is believed that a weak electrostatic bond is formed between the cationic drug and the polyanionic species. Thus, it is believed that the weak bond improves the ocular tolerability of the drug while having essentially no impact upon its bioavailability. While not intending to be bound in any way by theory, the experimental results provided herein in this and the other examples to be presented hereafter support this hypothesis.

Osmolality and dialysis studies were carried out with the memantine HCl/CMC model system to demonstrate that the polyanionic polymer does not significantly diminish the bioavailability of the neuroprotective amine. The osmotic pressure of a solution is a colligative property and as such can be a relative measure of free drug. This relationship is given by equation 1
Δπ=ΔCₘₑₘRT, (1)
where Δπ is the theoretical change in osmotic pressure for a given change in memantine concentration, ΔCₘₑₘ, if the individual memantine molecules are free and unbound. R is the universal gas constant and T the temperature in degrees Kelvin. By comparing the osmolality of memantine formulations, CMC containing memantine formulations and their respective placeboes the activity of the memantine can be inferred.

Osmolality measurements were carried out by freezing point depression osmometry. The results are presented in Table 2. The placebo establishes a baseline for the osmolality (the total number of particles per mass of solvent) of the solutions being studied, and the CMC placebo is used to establish the contribution of the CMC to the osmolality of the solution. Thus, the difference of 13 Osm/kg between the two solutions is attributed to the CMC. Comparison of the osmolality of a solution of 0.1% memantine to the placebo reveals that the memantine increases the osmolality of the solution by 10 Osm/kg, which compares well with the theoretical value of 9.3 based on the amount of CMC added and its molecular weight. The sum of the contribution of the CMC (13 Osm/kg) and the memantine (10 Osm/kg) would therefore predict an expected osmolality of about 23 Osm/kg higher than the placebo solution. The actual osmolality of the 0.1% memantine/CMC solution is 21 Osm/kg higher than the placebo, which is not significantly different than the theoretical expectation. This result suggests that the memantine HCl and CMC behave as essentially as individual particles, and not as a single complexed entity, in the 0.1% memantine/CMC solution. A similar result can be made by an analogous comparison between the 1% memantine solutions and the placebos. Hence, while not intending to be bound, or limit the scope of the invention in any way by theory, it is not expected that a weak interaction between memantine and CMC will reduce memantine's bioavailability.

TABLE 2
Osmolality Comparison of non-CMC Formulations and CMC
Formulations
Osmolality Δπ = ΔCₘₑₘRT

| | | CMC | 0.1% | 1.0% | 0.1% Mem | 1.0% Mem |
|---|---|---|---|---|---|---|
| | Placebo | placebo | Mem | Mem | CMC | CMC |
| Osm/Kg | 177 | 190 | 187 | 267 | 198 | 280 |
| Δπ | N/A | 13 | 10 | 90 | 21 | 103 |
| Theory | N/A | N/A | 9.3 | 93 | 23 | 106 |

The permeability of memantine through a dialysis membrane was studied as a model for the bioavailability of memantine. If the permeability of memantine through the dialysis membrane is equivalent for the CMC and non-CMC formulations, it is believed that the bioavailability of memantine will not be significantly different for the two types of formulations. These dialysis studies showed that the permeability of memantine from the CMC formulations through the dialysis membrane was equivalent to the permeability of memantine from the borate buffered non-CMC formulation (FIG. 3) through the membrane. In these studies the CMC containing or the non-CMC containing formulations of memantine were placed inside a dialysis bag. The bag was then submerged in a borate buffered formulation placebo reservoir and the appearance of memantine in the reservoir as a function of time was measured. The rate of appearance in the reservoir, drug permeation, is given by equation 2

$$\frac{ⅆ M}{ⅆ t} = \frac{2 π hP}{ln \frac{{r}_{o}}{{r}_{i}}} * {a}_{mem} \qquad ( 2 )$$

where

$$\frac{ⅆ M}{ⅆ t}$$

is the rate of memantine appearance in the reservoir as a function of time, h the thickness of the dialysis bag, P the permeability of memantine in the dialysis membrane, rₒ the outer diameter of the dialysis bag, rᵢ the inner diameter, and aₘₑₘ the memantine activity. In this experiment, only dM/dt and aₘₑₘ are not constant. As such, any difference in the rate of memantine permeation is directly and linearly related to an activity difference. Conversely, if two compositions give similar or identical rates of memantine permeation, the (memantine) activities of those compositions are essentially the same. FIG. 3 clearly shows that the permeation of memantine from non-CMC formulations and CMC formulations is equivalent and as such the activity of memantine is equivalent. While not intending to be bound in any way by theory, or be limited in any way, this suggests that in terms of membrane permeability, memantine activity is essentially the same whether or not a polyanionic polymer is present. Thus, if both the osmolality and activity in terms of membrane permeability are essentially unchanged for memantine in the presence of a polyanionic polymer, it is reasonable to believe that the polyanionic polymer will have a negligible effect on the bioavailability. While not intending to limit the scope of the invention in any way, the bioavailability data presented hereafter supports this conclusion.

### EXAMPLE 2

An initial toxicology screen included borate buffered, isotonic memantine HCl (0.05% to 1.0% w/v) solutions preserved with 20 ppm BAK (Table 1). Additionally, formulations containing 0.5% and 1.0% w/v memantine HCl in 0.5% sodium carboxymethylcellulose (CMC) vehicle (Table 2) were tested. A one day dose escalation study was conducted to up-titrate to the highest acceptable dose. Rabbits where dosed with 35 μL of formulation to the cul-de-sac and irritation was ranked as none, slight, mild, moderate or severe. The irritation score was a sum of lacrimation, chemosis, hyperemia and tolerability all scored from none to severe. The borate buffered placebo showed no irritation. However, the borate buffered memantine formulation showed slight irritancy at 0.1%, mild at 0.5% and moderate at 1.0%. The CMC placebo showed no irritation and the formulation showed only slight irritation at 1.0% memantine. Thus, the CMC formulation has the same irritation as the non-CMC formulation at a log unit higher memantine concentration. While not intending to be bound or limited in any way by theory, it is unexpected that the CMC would limit the irritation, but have a negligible effect on the predicted bioavailability, as assessed by the dialysis study.

This study was followed up by a five day toxicology study which included borate buffered formulations ranging from 0.1 to 0.75% memantine, CMC based formulations ranging from 0.1 to 1.0% memantine and the relevant placebos. All formulations were preserved with 20 ppm BAK. The formulations were dosed with 35 μL drops topically to the cul-de-sac of rabbits twice a day for 1 week. The rabbits were evaluated by gross clinical observation of irritation, ranked as none, slight, mild, moderate or severe. The irritation score was a sum of lacrimation, chemosis, hyperemia and tolerability all scored from none to severe. The borate buffered memantine formulations displayed a dose dependent increase in severity and frequency of ocular discomfort ranging from slight at 0.1% to moderate at 0.75% memantine HCl, while the CMC based formulations displayed only slight discomfort at 0.5% and 0.75% with mild discomfort at 1.0% mematine HCl. Thus, the tolerability of memantine is improved by a factor of about 5-8. Conjunctival hyperemia was slight to mild at 0.5% or higher for the borate solution, while a low frequency of slight hyperemia was observed for the CMC based formulation at 0.5 and 1.0% but not the other concentrations. Again, while not intending to be bound by theory, hyperemia is significantly reduced for the formulation containing the polyanionic polymer.

It was also important to assess the impact of the CMC on ocular bioavailability. A pharmacokinetic study was conducted to compare ocular concentrations after 0.05% non-CMC and 0.05% CMC formulations and to assess dose linearity of two CMC formulations (0.05% versus 0.25%). The formulations were dosed Female albino rabbits twice a day for 7 days to both eyes. Non-radiolabeled formulations were manufactured and spiked with ¹⁴C-Memantine HCl with a specific activity of 33.7 mCi/mmol. Final activity of the formulations ranged from 66.3 to 72.5 μCi/mL. After dosing the rabbits, six eyes per time point were sampled at predose, 30, 60, 120 and 240 minutes post dose. The conjunctiva, aqueous humor, cornea, iris-ciliary body, lens and sclera were assayed. Vitreous humor, retina and choroid were assayed only at predose, 30, 120 and 240 minutes post-dose. Tissue memantine concentrations were determined by tissue combustion with activity measurement by scintillation counter. The vitreous and aqueous humors were counted without combustion. Disintegrations per minutes were then correlated to tissue concentrations.

At 0.05% w/v memantine, the CMC and the non-CMC formulations displayed equivalent retinal concentrations. Retinal concentrations of the 0.25% memantine/CMC based formulation were approximately 4 fold higher than the retinal concentrations of the 0.05% formulations, thus indicating a nearly linear dose response. Thus, while not intending to limit the scope of the invention, or be bound in any way by theory, these results show that the irritancy mitigating affect of the CMC on memantine does not significantly reduce the bioavailability. While not intending to be limited or bound in any way by theory, these results indicate that a higher concentration of memantine may be used in a formulation while the irritation is the same or less than that previously observed at lower concentrations. Alternatively, while not intending to be limited in any way by theory, one may formulate a less irritating memantine composition by the addition of a polyanionic polymer.

### EXAMPLE 3

Compositions are prepared according to the procedure of Example 1 using the formulas described in Table 3.

TABLE 3

| | | Percent w/v | | | | | |
|---|---|---|---|---|---|---|---|
| Components | Function | 1 | 2 | 3 | 4 | 5 | 6 |
| Memantine | Active | 0.05 | 0.1 | 0.2 | 0.5 | 0.75 | 1.0 |
| HCl | | | | | | | |
| Sodium | Tonicity | 0.65 | 0.65 | 0.65 | 0.65 | 0.65 | 0.65 |
| Chloride | adjuster | | | | | | |
| Boric Acid | Buffering | 0.64 | 0.64 | 0.64 | 0.64 | 0.64 | 0.64 |
| | agent | | | | | | |
| Sodium Borate, | Buffering | 0.16 | 0.16 | 0.16 | 0.16 | 0.16 | 0.16 |
| Decahydrate | agent | | | | | | |
| Stabilized | Preservative | 0.005 | 0.005 | 0.005 | 0.005 | 0.005 | 0.005 |
| Oxychloro | | | | | | | |
| Complex | | | | | | | |
| Sodium CMC | Polyanionic | 0.5 | 0.5 | 0.5 | 0.5 | 0.5 | 0.5 |
| (Type 7LFH) | polymer | | | | | | |
| Hydrochloric | pH | 7.4 | 7.4 | 7.4 | 7.4 | 7.4 | 7.4 |
| Acid | adjustment | | | | | | |
| Sodium | pH | 7.4 | 7.4 | 7.4 | 7.4 | 7.4 | 7.4 |
| Hydroxide | adjustment | | | | | | |
| Purified Water | Vehicle | Q.S. | Q.S. | Q.S. | Q.S. | Q.S. | Q.S. |

### EXAMPLE 4

Compositions are prepared according to the procedure of Example 1 using the formulas described in Table 4.

TABLE 4

| | | Percent w/v | | | | | |
|---|---|---|---|---|---|---|---|
| Components | Function | 1 | 2 | 3 | 4 | 5 | 6 |
| Memantine | Active | 0.05 | 0.1 | 0.2 | 0.5 | 0.75 | 1.0 |
| HCl | | | | | | | |
| Sodium | Tonicity | 0.72 | 0.72 | 0.72 | 0.72 | 0.72 | 0.72 |
| Chloride | adjuster | | | | | | |
| Sodium Citrate | Buffering | 0.45 | 0.45 | 0.45 | 0.45 | 0.45 | 0.45 |
| | agent | | | | | | |
| Citric Acid | Buffering | 0.01 | 0.01 | 0.01 | 0.01 | 0.01 | 0.01 |
| | agent | | | | | | |
| Benzalkonium | Preservative | 0.005 | 0.005 | 0.005 | 0.005 | 0.005 | 0.005 |
| Chloride | | | | | | | |
| Carbomer 940 | Polyanionic | 0.2 | 0.2 | 0.2 | 0.2 | 0.2 | 0.2 |
| | Polymer | | | | | | |
| Hydrochloric | pH | 6.3 | 6.3 | 6.3 | 6.3 | 6.3 | 6.3 |
| Acid | adjustment | | | | | | |
| Sodium | pH | 7.4 | 7.4 | 7.4 | 7.4 | 7.4 | 7.4 |
| Hydroxide | adjustment | | | | | | |
| Purified Water | Vehicle | Q.S. | Q.S. | Q.S. | Q.S. | Q.S. | Q.S. |

### EXAMPLE 5

Compositions are prepared according to the procedure of Example 1 using the formulas described in Table 5.

TABLE 5

| | | Percent w/v | | | | | |
|---|---|---|---|---|---|---|---|
| Components | Function | 1 | 2 | 3 | 4 | 5 | 6 |
| Memantine | Active | 0.05 | 0.1 | 0.2 | 0.5 | 0.75 | 1.0 |
| HCl | | | | | | | |
| Sodium | Tonicity | 0.65 | 0.65 | 0.65 | 0.65 | 0.65 | 0.65 |
| Chloride | adjuster | | | | | | |
| Boric Acid | Buffering | 0.64 | 0.64 | 0.64 | 0.64 | 0.64 | 0.64 |
| | agent | | | | | | |
| Sodium Borate, | Buffering | 0.16 | 0.16 | 0.16 | 0.16 | 0.16 | 0.16 |
| Decahydrate | agent | | | | | | |
| Chlorobutanol | Preservative | 0.2 | 0.2 | 0.2 | 0.2 | 0.2 | 0.2 |
| Sodium CMC | Polyanionic | 0.5 | 0.5 | 0.5 | 0.5 | 0.5 | 0.5 |
| (Type 7LFH) | Polymer | | | | | | |
| Hydrochloric | pH | 7.4 | 7.4 | 7.4 | 7.4 | 7.4 | 7.4 |
| Acid | adjustment | | | | | | |
| Sodium | pH | 7.4 | 7.4 | 7.4 | 7.4 | 7.4 | 7.4 |
| Hydroxide | adjustment | | | | | | |
| Purified Water | Vehicle | Q.S. | Q.S. | Q.S. | Q.S. | Q.S. | Q.S. |

### EXAMPLE 6

Compositions are prepared according to the procedure of Example 1 using the formulas described in Table 6.

TABLE 6

| | | Percent w/v | | | | | |
|---|---|---|---|---|---|---|---|
| Components | Function | 1 | 2 | 3 | 4 | 5 | 6 |
| Memantine | Active | 0.05 | 0.1 | 0.2 | 0.5 | 0.75 | 1.0 |
| HCl | | | | | | | |
| Sodium | Tonicity | 0.72 | 0.72 | 0.72 | 0.72 | 0.72 | 0.72 |
| Chloride | adjuster | | | | | | |
| Sodium Citrate | Buffering | 0.45 | 0.45 | 0.45 | 0.45 | 0.45 | 0.45 |
| | agent | | | | | | |
| Citric Acid | Buffering | 0.01 | 0.01 | 0.01 | 0.01 | 0.01 | 0.01 |
| | agent | | | | | | |
| Chlorobutanol | Preservative | 0.2 | 0.2 | 0.2 | 0.2 | 0.2 | 0.2 |
| Carbomer 940 | Polyanionic | 0.2 | 0.2 | 0.2 | 0.2 | 0.2 | 0.2 |
| | Polymer | | | | | | |
| Hydrochloric | pH | 6.3 | 6.3 | 6.3 | 6.3 | 6.3 | 6.3 |
| Acid | adjustment | | | | | | |
| Sodium | pH | 7.4 | 7.4 | 7.4 | 7.4 | 7.4 | 7.4 |
| Hydroxide | adjustment | | | | | | |
| Purified Water | Vehicle | Q.S. | Q.S. | Q.S. | Q.S. | Q.S. | Q.S. |

### EXAMPLE 7

A polyanionic polymer containing composition according to one of examples 1-6 is administered twice daily to a person suffering from glaucoma for a prolonged period of time. Irritation is below a tolerable level throughout the treatment, and the rate of vision loss is significantly reduced.

1. An aqueous solution comprising an adamantane-based neuroprotective amine and a polyanionic polymer.

2. The aqueous solution of claim 1 comprising memantine, rimantadine, amantadine.

3. The aqueous solution of claim 1 comprising memantine.

4. The aqueous solution of claim 1 comprising a cationic preservative.

5. The aqueous solution of claim 1 wherein the polyanionic polymer is selected from the group consisting of carboxymethylcellulose, hyaluronic acid, carboxymethylamylose, poly(methacrylic acid) derivatives, polyphospazene derivatives, poly(aspartic acid), acidic gelatin, alginic acid and polyacrylic acid.

6. The aqueous solution of claim 1 comprising carboxymethylcellulose.

7. The aqueous solution of claim 1 comprising a member of the group consisting of benzalkonium chloride, stabilized oxychloro complexes, phenylmercuric acetate, chlorobutanol, benzyl alcohol, parabens, and thimerosal

8. The aqueous solution of claim 1 which comprises benzalkonium chloride.

9. The aqueous solution of claim 1 which comprises from 0.05 to 5% memantine.

10. The aqueous solution of claim 1 which comprises from 0.1 to 2% memantine.

11. The aqueous solution of claim 1 comprising from 0.1 to 5% carboxymethylcellulose.

12. The aqueous solution of claim 1 comprising from 0.05% to 2% memantine and from 0.1% to 5% sodium carboxymethylcellulose.

13. The aqueous solution of claim 1 comprising from 0.05% to 2.5% memantine, from 0.1% to 5% sodium carboxymethylcellulose, and from 10 ppm to 200 ppm benzalkonium chloride.

14. The aqueous solution of claim 1 comprising from about 0.5% to about 2% memantine, about 0.5% sodium carboxymethylcellulose, and about 20 ppm benzalkonium chloride.

15. A method comprising administering an effective amount of neuroprotective compound comprising an adamantyl moiety and an amine moiety in an aqueous composition comprising an effective amount of a soluble polyanionic polymer to the eye of a mammal suffering from a disease or condition selected from the group consisting of glaucoma, ocular hypertension, nystagmus, proliferative vitreal retinopathy or ocular neurodegenerative diseases.

16. The method of claim 15 comprising amantadine.

17. The method of claim 15 comprising rimantadine.

18. The method of claim 15 comprising memantine.

19. The method of claim 15 wherein the polyanionic polymer is selected from the group consisting of carboxymethylcellulose, hyaluronic acid, carboxymethylamylose, poly(methacrylic acid) derivatives, polyphospazene derivatives, poly(aspartic acid), acidic gelatin, alginic acid and polyacrylic acid.

20. The method of claim 15 wherein the composition comprises carboxymethylcellulose.

21. The method of claim 15 wherein the composition comprises memantine and sodium carboxymethylcellulose.

22. The method of claim 15 wherein the composition comprises benzalkonium chloride.

23. The method of claim 15 wherein the composition comprises about 0.5% memantine, from 0.1% to 1.5% carboxymethylcellulose, and said composition further comprises an effective amount of benzalkonium chloride.

24. The method of claim 15 wherein the composition comprises from 0.5% to 2% memantine, an effective amount of carboxymethylcellulose, and said composition further comprises an effective amount of benzalkonium chloride.

25. The method of claim 15 wherein the composition comprises about 1% memantine, from 0.1% to 1.5% carboxymethylcellulose, and said composition further comprises an effective amount of benzalkonium chloride.

26. An eye drop product comprising
an aqueous solution comprising an effective amount of an adamantane-based neuroprotective amine and an effective amount of a polyanionic polymer, and a package for dispensing said solution in the form of drops suitable for administration to an eye of a mammal.

27. The product of claim 26 comprising rimantadine or amantadine.

28. The product of claim 26 comprising memantine.

29. The product of claim 26 wherein the polyanionic polymer is selected from the group consisting of carboxymethylcellulose, hyaluronic acid, carboxymethylamylose, poly(methacrylic acid) derivatives, polyphospazene derivatives, poly(aspartic acid), acidic gelatin, alginic acid and polyacrylic acid.

30. The product of claim 26 which comprises carboxymethylcellulose.

31. The product of claim 26 which comprises benzalkonium chloride.

32. The product of claim 26 wherein said aqueous solution comprises from 0.2% to 3% memantine, from 0.5% to 4% sodium carboxymethylcellulose, and an effective amount of benzalkonium chloride.

33. The product of claim 26 wherein said aqueous solution comprises from 0.5% to 3.5% memantine, from 0.4% to 4.5% sodium carboxymethylcellulose, and 20 ppm benzalkonium chloride.

34. The method of claim 15 wherein migration or proliferation of retinal pigment epithelium or glial cells causes or contributes to the cause of said disease or condition.

35. The method of claim 34 comprising memantine.

36. The method of claim 15 wherein said neuroprotective compound consists essentially of memantine.

37. The method of claim 34 wherein said neuroprotective compound consists essentially of memantine.

38. The method of claim 34 wherein said disease or condition is selected from the group consisting of non-exudative age related macular degeneration, exudative age related macular degeneration, choroidal neovascularization, acute macular neuroretinopathy, cystoid macular edema, diabetic macular edema, Behcet's disease, diabetic retinopathy, retinal arterial occlusive disease, central retinal vein occlusion, uveitic retinal disease, retinal detachment, trauma, conditions caused by laser treatment, conditions caused by photodynamic therapy, photocoagulation, radiation retinopathy, epiretinal membranes, proliferative diabetic retinopathy, branch retinal vein occlusion, anterior ischemic optic neuropathy, non-retinopathy diabetic retinal dysfunction, and retinitis pigmentosa.

39. A composition comprising an adamantane-based neuroprotective amine and a polyanionic polymer.

40. The composition of claim 39 comprising memantine.

41. The composition of claim 40 comprising carboxymethylcellulose.

42. The method of claim 15 wherein said disease or condition comprises nystagmus.

43. The method of claim 15 wherein said disease or condition comprises glaucoma or ocular hypertension.
